SEQUENCE LISTING (29 sequences)

SEQ ID NO: 1 - protein, 574 aa, respiratory syncytial virus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 2 - protein, 306 aa, respiratory syncytial virus
MSKNKDQRTAKTLERTWDTLNHLLFISSCLYKLNLKSVAQITLSILAMIISTSLIIAAIIFIASANHKVTPTTAIIQDATSQIKNTTPTYLTQNPQLGISPSNPSEITSQITTILASTTPGVKSTLQSTTVKTKNTTTTQTQPSKPTTKQRQNKPPSKPNNDFHFEVFNFVPCSICSNNPTCWAICKRIPNKKPGKKTTTKPTKKPTLKTTKKDPKPQTTKSKEVPTTKPTEEPTINTTKTNIITTLLTSNTTGNPELTSQMETFHSTSSEGNPSPSQVSTTSEYPSQPSSPPNTPRQSPPNTPRQ

SEQ ID NO: 3 - protein, 17 aa, respiratory syncytial virus
KANAITILTAVTFCFAS

SEQ ID NO: 4 - protein, 18 aa, respiratory syncytial virus
TILTAVTFCFASGQNITE

SEQ ID NO: 5 - protein, 18 aa, respiratory syncytial virus
GQNITEEFYQSTCSAVSK

SEQ ID NO: 6 - protein, 18 aa, respiratory syncytial virus
EFYQSTCSAVSKGYLSAL

SEQ ID NO: 7 - protein, 18 aa, respiratory syncytial virus
SVITIELSNIKENKCNGT

SEQ ID NO: 8 - protein, 18 aa, respiratory syncytial virus
IKQELDKYKNAVTELQLL

SEQ ID NO: 9 - protein, 18 aa, respiratory syncytial virus
KYKNAVTELQLLMQSTPP

SEQ ID NO: 10 - protein, 18 aa, respiratory syncytial virus
NKAVVSLSNGVSVLTSKV

SEQ ID NO: 11 - protein, 18 aa, respiratory syncytial virus
RLLEITREFSVNAGVTTP

SEQ ID NO: 12 - protein, 18 aa, respiratory syncytial virus
REFSVNAGVTTPVSTYML

SEQ ID NO: 13 - protein, 18 aa, respiratory syncytial virus
PITNDQKKLMSNNVQIVR

SEQ ID NO: 14 - protein, 17 aa, respiratory syncytial virus
KLMSNNVQIVRQQSYSI

SEQ ID NO: 15 - protein, 18 aa, respiratory syncytial virus
EVLAYVVQLPLYGVIDTP

SEQ ID NO: 16 - protein, 18 aa, respiratory syncytial virus
VQLPLYGVIDTPCWKLHT

SEQ ID NO: 17 - protein, 18 aa, respiratory syncytial virus
TDRGWYCDNAGSVSFFPQ

SEQ ID NO: 18 - protein, 18 aa, respiratory syncytial virus
YDCKIMTSKTDVSSSVIT

SEQ ID NO: 19 - protein, 18 aa, respiratory syncytial virus
KNRGIIKTFSNGCDYVSN

SEQ ID NO: 20 - protein, 18 aa, respiratory syncytial virus
YYVNKQEGKSLYVKGEPI

SEQ ID NO: 21 - protein, 18 aa, respiratory syncytial virus
LIAVGLLLYCKARSTPVT

SEQ ID NO: 22 - protein, 18 aa, respiratory syncytial virus
SLGAIVSCYGKTKCTASN

SEQ ID NO: 23 - protein, 14 aa, respiratory syncytial virus
DFHFEVFNFVPCSI

SEQ ID NO: 24 - protein, 18 aa, respiratory syncytial virus
SNNPTCWAICKRIPNKKP

SEQ ID NO: 25 - protein, 15 aa, respiratory syncytial virus
FEVFNFVPCSICSNN

SEQ ID NO: 26 - protein, 16 aa, respiratory syncytial virus
FNFVPCSICSNNPTCW

SEQ ID NO: 27 - protein, 16 aa, respiratory syncytial virus
SICSNNPTCWAICKRI

SEQ ID NO: 28 - protein, 15 aa, respiratory syncytial virus
WAICKRIPNKKPGKK

SEQ ID NO: 29 - protein, 15 aa, respiratory syncytial virus
ICKRIPNKKPGKKTT

(19) United States
(12) Patent Application Publication
(10) Pub. No.: US 2005/0249744 A1
(43) Pub. Date: Nov. 10, 2005

(54) Mhc class II haplotype specific immunodominancy of peptides derived from rsv fusion (f) and attachement (g) proteins

(76) Inventors: Cecile Antoinette Carola Maria Van Els, Bilthoven (NL); Gerarda Maria Van Bleek, Houten (NL); Marie-Claire Josefa Pia Boog, Nunspeet (NL)

Correspondence Address: MCKENNA LONG & ALDRIDGE LLP, 1900 K STREET, NW, WASHINGTON, DC, 20006, US

(21) Appl. No.: 10/518,341

(22) PCT Filed: Jun. 20, 2003
(86) PCT No.: PCT/NL03/00454

§ 371 Date: Dec. 17, 2004

(30) Foreign Application Priority Data
Jun. 20, 2002 (EP) 02077461.8

(51) Int. Cl. C12Q 1/70; A61K 39/12

(52) U.S. Cl. 424/186.1; 435/5

(57) ABSTRACT

The present invention relates immunodominant peptides derived from human respiratory syncytial virus (H-RSV) that may be used in ex vivo diagnosis of immune responses to H-RSV The immunodominant peptides are derived from the H-RSV Fusion (F) and Attachment (G) proteins and are capable of inducing an antigen specific CD4⁺ T cell response ex vivo in a MHC class 11 haplotype restricted manner. The immunodominant H-RSV-derived peptides may further be used in methods for vaccination against H-RSV, preferably in a MHC class 11 haplotype specific manner.

### FIELD OF THE INVENTION

The present invention relates immunodominant peptides derived from human respiratory syncytial virus (H-RSV) that may be used in ex vivo diagnosis of immune responses to H-RSV, preferably in a MHC class II haplotype specific manner. The immunodominant peptides are preferably derived from the H-RSV Fusion (F) and Attachment (G) proteins. The invention further relates to the use of these immunodominant H-RSV-derived peptides for vaccination against H-RSV, preferably in a MHC class II haplotype specific manner.

### BACKGROUND OF THE INVENTION

Human respiratory syncytial virus (H-RSV), classified in the genus Pneumoviruses of the family of Paramyxoviridae, is a major cause of lower respiratory disease in young infants, immunocompromised individuals and the elderly. No safe and effective licensed H-RSV vaccine is currently available. In an early vaccine trial with a formalin inactivated alum-precipitated H-RSV vaccine (FI-RSV) given intramuscularly, an enhancement of disease occurred in vaccinees upon subsequent exposure to the natural virus. This enhanced illness was characterised by bronchiolitis, hypoxemia and pneumonia and infiltration into the lungs of lymphocytes, neutrophils and some eosinophils. This cellular infiltration suggested that immune-mediated injury can contribute to the pathogenicity of RSV disease.

Primary infection with RSV can cause lower respiratory tract disease in young infants manifesting as a pneumonia or bronchiolitis. The disease is associated with the inflammatory response to infection likely involving the production of cytokines and chemokines by lung epithelial cells and immune cells that are recruited to the lungs. The T cell response is an essential component of the immune response needed for viral clearance from the lungs (Alwan et al., 1992, Clin. Exp. Immunol. 8: 527-536; Graham et al., 1991, J.Clin. Invest. 88:1026-1033; Fishaut M. et al., 1980, J. Pediatr. 96:179-186). Antibodies against F and G proteins are generated during RSV infection but even in the presence of high levels of virus neutralising antibodies reinfections occur (Hall et al., 1991, J. Inf. Dis. 163: 693-698) and antibodies are not needed for viral clearance (Graham et al., 1991, J. Virol. 65: 4936-4942). Also, vaccination with the FI-RSV vaccine induced high titres of RSV specific antibodies in vaccinees, yet caused more severe clinical disease upon natural infection. While in RSV infections T cell responses are required for clearance of RSV from the lung it has been shown in murine studies that CD4 as well as CD8 T cells can be responsible for enhanced lung pathology (Cannon et al., 1988, J.Exp. Med. 168:1163-1168; Alwan et al., 1994, J. Exp. Med. 179: 81-89). Severe pneumonia with extensive influx of eosinophils into the lungs can be elicited in mice by Th2 cells specific for RSV-G, that are primed during vaccination with FI-RSV before intra-nasal challenge with life RS-virus (Waris et al., 1996, J. Virol. 70: 2852-2860; Openshaw et al., 1992, Int. Immunol. 4: 493-500). This strong eosinophilic inflammation is also observed after RSV challenge of mice vaccinated with a vaccinia recombinant expressing solely the G protein of RSV. One peptide corresponding to residues 183-195 in the G protein of RSV appears to be recognised by Balb/c specific Th2 cells (Varga et al., 2000, J. Immunol. 165: 6487-6495). CD8 T cell responses against epitopes derived from G protein have not been observed in BALB/c mice nor in man. CD4 T cells in mice vaccinated with a vaccinia recombinant expressing the F protein of RSV that are subsequently challenged with life virus are Th1 (Alwan et al., 1993, J. Immunol. 150: 5211-5218). CD8 T cell responses against F are common in mice of different MHC types (Chang et al., 2001, J. Immunol. 167:4254-4260). In the Balb/c model a CD8 T cell response has been shown to regulate the outcome of CD4 T cell responses preventing enhanced disease (Srikiatkhachorn and Braciale, 1997, J. Exp. Med. 186: 421-432). Whether the insights obtained from the murine model can be translated to the human situation has to be evaluated.

In PBMC's from healthy adults as well as from diseased infants, CD8 T cell responses and CD4 T cell responses can be visualised. Some information on viral protein/peptide recognition by human CD4 T cells and CD8 T cells has been uncovered in the art. E.g. in Levely et al. (1991, J. Virol. 65: 3789-3796) CD4 T cell responses against some subdomains of the F protein have been described using T cell proliferation assays. A dominant antigenic peptide was characterised encompassing amino acid residues 338-355 of the F protein. The response against this peptide was HLA-DR restricted, but the presenting MHC molecule was not further identified. Moreover, the peptide was recognised by different donors that did not share one single MHC class II molecule. Goulder et al. (2000, J. Virol. 74: 7694-7697) describe an H-RSV specific cytotoxic T cell epitope that is HLA class I (HLA B7) restricted and that is derived from the H-RSV nucleoprotein. Brandenburg et al. (2000, J. Virol 74: 10240-10244) describe HLA class I restricted cytotoxic T cell epitopes derived from the H-RSV F protein. A peptide spanning amino acids 118-126 of H-RSV F protein proved to be HLA B57 restricted in one patient and a peptide spanning amino acids 551-559 of H-RSV F protein proved to be HLA C12 restricted in another patient.

There is, however, still a need for further characterisation of the MHC class II haplotype specific immunodominancy of peptides derived from the F and G proteins of RSV. Such detailed knowledge about antigenic peptides may be used for the development of subunit vaccines and it may further enable the use of tools, like MHC class II tetramers, to monitor ex vivo virus specific immune responses in infected patients or evaluate correlates of protection in vaccinated individuals.

### DESCRIPTION OF THE INVENTION

The present invention is based on the discovery and characterisation of immunodominant peptides derived from the Fusion (F) and Attachment (G) proteins of H-RSV that are recognised by autologous CD4 T cells in the context of a panel of MHC class II molecules that are expressed most frequently within the Caucasian population. Antigenic peptides are found in both G and F protein. In most donors, CD4 cells recognise more than one peptide within the F protein. Several peptide antigens are productively presented in the context of more than one MHC class II molecule, while at the level of detection of the direct elispot assays performed, there are also peptides that are MHC class II haplotype specific. Most peptides are presented by HLA DR molecules. In general donors that share MHC class II molecules recognise the same pattern of peptides. The present invention thus relates to diagnostic, prophylactic and therapeutic methods that are based on the MHC class II haplotype specific antigenicity of H-RSV F or G protein-derived peptides.

In a first aspect the invention relates to method for ex vivo diagnosis of MHC class II haplotype specific immune responses to H-RSV antigens in a subject. The method comprises the steps of: (a) determining the MHC class II haplotype of the subject; (b) providing a composition comprising peripheral blood mononuclear cells (PBMC's) from the subject; (c) mixing the composition comprising PBMC's with a peptide comprising at least 9 contiguous amino acids from an amino acid sequence selected from Table 1 that matches the MHC class II haplotype of the subject in accordance with Table 1; and, (d) determining the response of the PBMC's to the peptide.

In the method, the MHC class II haplotype of the subject is determined using any one of a number of methods well known in the art, (see e.g. Coligan et al., 1994, In: Coico R, ed. Current protocols in immunology. Vol. 2: John Wiley & Sons, Inc., Chapter 7: Immunologic studies in humans). Similarly, the composition comprising PBMC's is obtained from the (human) subject using a variety of methods well known in the art (see e.g. Coligan et al., 1994, In: Coico R, ed. Current protocols in immunology. Vol. 2: John Wiley & Sons, Inc.,: 711-712). Blood samples are usually processed to remove erythrocytes and platelets (e.g., by aphaeresis, Ficoll density gradient centrifugation and/or red blood cell lysis or other such methods known to one of skill in the art) and the remaining PBMC sample, which includes the T-cells of interest, as well as B-cells, macrophages and dendritic cells, may be used directly in the assay. The composition comprising PBMC's may e.g. consist of the PBMC bulk that is obtainable by from blood obtained from the subject. For higher yields of PBMCs, the subject from whom the PBMCs are obtained may be given a pre-treatment with GM-CSF for mobilising mononuclear cell subpopulations. The PBMC composition may further be enriched for specific subsets of mononuclear cells, preferably T cells, more preferably CD4⁺ T cells. The PBMC composition may be enriched for T cells, or in particular for CD4⁺ T cells, by methods known in the art, such e.g. by expansion of T cells or CD4⁺ T cells as described e.g. in Coligan et al. (1994, In: Coico R, ed. Current protocols in immunology. Vol. 2: John Wiley & Sons, Inc., 1994: 711-94).

In step (c) of the diagnostic methods of the invention a selected peptide derived from the H-RSV F and/or G proteins is mixed with the composition comprising the PBMC's. The H-RSV F or G protein-derived peptides of the invention may be obtained as described below. The peptide to be mixed with the composition comprising the PBMC's of a given subject are selected to match the MHC class II haplotype of the subject. Table 1 summarises for each of the major MHC class II haplotypes that are prevalent in the caucasian population, the H-RSV F and G protein derived peptides that are recognised by the particular MHC class II haplotype, as measured by IFN-γ production in a direct elispot assay. Thus, in the method of the invention, subsequent to having established the MHC class II haplotype of a subject, the composition comprising the PBMC's form the a subject is brought into contact with one or more (poly)peptides that comprise an amino acid sequence of at least 9, 10, 11 or 12 contiguous amino acids from an amino acid sequence listed in Table 1 as being recognised by the MHC class II haplotype of the subject. Peptides and polypeptides of the invention are further described herein below. Preferably the peptides are brought into contact with the composition comprising the PBMC's at a concentration that ranges from 1 nM to 100 μM, more preferably 10 nM to 50 μM, most preferably 100 nM to 10 μM. The amount of PBMC's to be brought into contact with the peptide depends on the assay format used for detecting the PBMC response. Various such assays are described below and the amount of PBMC's to be used in these assays are known to the skilled person.

Finally, in step (d) of the method, the response of the PBMC's to the H-RSV antigenic peptide(s) is determined. The antigen specific response of the PBMC's may be determined using a number of methods available in the art (see e.g. Coligan et al., 1994, In: Coico R, ed. Current protocols in immunology. Vol. 2: John Wiley & Sons, Inc., Chapter 7: Immunologic studies in humans). Preferably, the antigen specific response of the PBMC's is determined by determining the proliferation of the PBMC's in response to the presence of the H-RSV antigenic peptides. More preferably, the proliferation of T cells in response to the presence of the H-RSV peptide(s) is determined. The proliferation of T cells in response to the presence of the H-RSV peptide(s) is preferably determined directly in the composition comprising the PBMC's, i.e. without pre-expansion of the T cells. Alternatively, the T cells or more specifically, the CD4⁺ T cells may be enriched or pre-expanded to increase the sensitivity of the diagnostic method of the invention. In a further preferred method, the proliferation of CD4⁺ T cells (in the composition comprising PBMC's) in response to the presence of the H-RSV peptide(s) is determined. The proliferation or response of the CD4⁺ T cells may be determined using a variety of methods available in the art, (see e.g. Coligan et al., 1994, In: Coico R, ed. Current protocols in immunology. Vol. 2: John Wiley & Sons, Inc., Chapter 7: Immunologic studies in humans). In a preferred method the proliferation or response of the PBMC's, in particular the CD4⁺ T cells is determined by measuring the concentration of a “soluble protein factor” that is secreted by a PBMC or T-cell in response to antigenic stimulation. A variety of secreted soluble protein factors can be detected by the assays disclosed herein. The soluble factors may be cytokines, lymphokines or chemokines. Typically this secreted factor is a lymphokine, such as enumerated below. As a result of the increased sensitivity of the assay, factors secreted by rare T-cells which occur in low frequency can be detected. Factors which are detected by this method include, but are not limited to lymphokines, cytokines and chemokines such as for example, IFN-γ, TNF-α, IL-2, IL-3, IL-4, IL-5, IL-10, IL-13, TGF-β, RANTES, and GM-CSF.

A variety of assay formats can be used to detect the increased levels of secreted factors produced by the assay described herein. Suitable assays include both solid phase (heterogeneous) and non-solid phase (homogeneous) protocols. The assays can be run using competitive or non-competitive formats, and using a wide variety of labels, such as radioisotopes, enzymes, fluorescers, chemiluminescers, spin labels, and the like. Such methods include, but are not limited to enzyme-linked immunosorbent assays (ELISA), both direct and reverse formats, and other solid phase assays. It will be recognised that negative controls, i.e., samples run without added antigen, and positive controls, i.e., samples run with antigens, such as tetanus toxoid, known to elicit lymphokine secretion from T-cells will be run as necessary under otherwise duplicative conditions to validate the assay results.

Some assays rely on heterogeneous protocols where a ligand complementary to the secreted factor (such as antibody against the secreted factor) is bound to a solid phase which is used to capture the secreted factor. The ligand may be conveniently immobilised on a variety of solid phases, such as dipsticks, particulates, microspheres, magnetic particles, test tubes, microtiter wells, and plastics, nitrocellulose or nylon membranes and the like, including polyvinyl difluoride (PVDF) (e.g., 96 well plate with a PVDF membrane base (Millipore MAIPS45-10))and ELISA grade plastic. The captured factor can then be detected using the non-competitive “sandwich” technique where a directly or indirectly labelled second ligand for the factor is exposed to the washed solid phase. Such assay techniques are well known and well described in both the patent and scientific literature. See, e.g., U.S. Pat. Nos. 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; and 4,098,876. Enzyme-linked immunosorbent assay (ELISA) methods are described in detail in U.S. Pat. Nos. 3,791,932; 3,839,153; 3,850,752; 3,879,262; and 4,034,074. ELISA assays detect very low titres of secreted factors. Also see, “Enzyme immunohistochemistry” in Practice and Theory of Enzyme Immunoassays, P. Tijssen (Elsevier 1985).

A commonly used assay format is the antibody capture assay. The general protocol is simple: a ligand, e.g., an unlabeled antibody for the secreted factor, is immobilised on a solid phase, and the secreted factor is allowed to bind to the immobilised antibody. The bound secreted factor is then detected by using a labelled secondary reagent that will specifically bind to the captured factor (“direct sandwich assay”). Alternatively, the secondary reagent will not be labelled, but will be detected by subsequent binding to labelled tertiary binding reagent complementary to the second binding reagent (“indirect sandwich assay”). The strength of signal from the bound label allows the determination of the amount of secreted factor present in the sample and this in turn allows the quantification of the number of activated T-cells present in the sample. As will be recognised by the skilled person, the sandwich assay can be used to detect any secreted factor which has two epitopes, each of which can be recognised by the specific binding pair members. Choosing appropriate capture and detection antibody pairs permits the application of this assay to the detection of T cells secreting a variety of soluble factors. A list of antibody pairs which can be used in this assay is inter alia presented in U.S. Pat. No. 6,218,132.

Alternatively, the proliferation or response of the T cells may be determined by intracellular cytokine staining as e.g. described by Murali-Krishna et al. (1998, Immunity 8: 177-187).

A preferred method for determining the proliferation or response of the CD4⁺ T cells comprises measuring the IFN-γ production by the CD4⁺ T cells in response to the presence of the H-RSV peptide(s). Again, methods for measuring antigen induced IFN-γ production by the CD4⁺ T cells are well known in the art (see e.g. Coligan et al., 1994, In: Coico R, ed. Current protocols in immunology. Vol. 2: John Wiley & Sons, Inc., Chapter 6: Cytokines)., and include e.g. a method wherein IFN-γ production is measured in a elispot assay, as described in the Examples herein. Preferably, the IFN-γ production is measured in a direct elispot assay, whereby “direct” is understood to mean directly on the composition of PBMC's obtained from the subject, without pre-expansion of the T cells or CD4⁺ T cells. Likewise, the other above-mentioned lymphokines and cytokines may be measured in a direct elispot assay.

The diagnostic methods of the invention may be used for a variety of reasons on different subjects. The methods may e.g. be used on a subject undergoing a lower respiratory tract (LRT) disease as manifested by pneumonia and/or bronchitis. In such subjects, the method may be used to determine the involvement of H-RSV in the LRT disease, e.g. to distinguish between infections with H-RSV, influenza virus and/or human pneumovirus. Having established the diagnosis of an H-RSV infection in a given subject, the diagnostic methods of the invention may further be used to monitor the immune response to H-RSV antigens in the subject undergoing an H-RSV infection. The methods may further be used to determine the status of a subject's immune response to H-RSV in a subject having undergone an infection with H-RSV, i.e. in a subject that no longer shows any clinical symptoms of H-RSV infection. Finally, the methods of the invention may be used to determine the status of a subject's immune response to H-RSV in a subject having been vaccinated against H-RSV. In particular, the methods may be used to evaluate correlates of protection in individuals having been vaccinated against H-RSV.

In a further aspect, the invention relates to a method for MHC class II haplotype specific vaccination of a subject against H-RSV, the method comprising the steps of: (a) determining the MHC class II haplotype of the subject; and, (b) administering to the subject a pharmaceutical composition comprising a peptide comprising at least 9, 10, 11, or 12 contiguous amino acids from an amino acid sequence selected from Table 1, whereby the amino acid sequence matches the MHC class II haplotype of the subject in accordance with Table 1. In this method, the MHC class II haplotype of the subject is determined by known methods as described above. Method for preparing the peptides to be used in this method, as well as pharmaceutical composition comprising these peptides and methods for their preparation are described herein below. In a preferred method of vaccination, a pharmaceutical composition suitable for parenteral administration is administered parenterally, a pharmaceutical composition suitable for transdermal administration is administered transdermally, or a pharmaceutical composition suitable for inhalation is inhaled.

In yet another aspect, the invention relates to the use of a peptide comprising at least 9, 10, 11, or 12 contiguous amino acids from an amino acid sequence selected from Table 1, for the manufacture of a vaccine for MHC class II haplotype specific prophylaxis or therapy of H-RSV infection in a subject, whereby the amino acid sequence matches the MHC class II haplotype of the subject in accordance with Table 1. Preferably, the vaccine is a pharmaceutical composition suitable for parenteral or transdermal administration.

### Peptides for Use in the Methods of the Invention

The peptides of the invention contain an epitope that specifically recognised by MHC class II molecules in accordance with Table 1. The peptides of the invention thus bind the groove or cleft of the MHC class II molecule in question. The peptides of the invention typically comprise at least about 9, 10, 11, 12, 15, or 18 residues. In certain embodiments the peptides will not exceed about 150, 100 or 50 residues and typically will not exceed about 20 residues. Thus, a wide range of peptide sizes may be used in the present invention.

Particularly because the peptides to be used in the methods of the invention are relatively short, the peptides can be readily synthesised using known methods. For example, the peptides can be synthesised by the well-known Merrifield solid-phase synthesis method in which amino acids are sequentially added to a growing chain. See Merrifield (1963), J. Am. Chem. Soc. 85:2149-2156; and Atherton et al., “Solid Phase Peptide Synthesis,” IRL Press, London, (1989). Automatic peptide synthesisers are commercially available from numerous suppliers, such as Applied Biosystems, Foster City, Calif. Additional synthetic approaches for preparing the peptides of the invention are described in the Examples herein.

Alternatively, the F and G protein-derived peptides may be prepared as part of larger polypeptides comprising one or more of the peptides of the invention. Such larger polypeptides be prepared using well-known recombinant techniques in which a nucleotide sequence encoding the polypeptide of interest is expressed in cultured cells such as described in Ausubel et al., Current Protocols in Molecular Biology, Greene Publishing and Wiley-Interscience, New York (1987) and in Sambrook and Russell (2001) “Molecular Cloning: A Laboratory Manual (3^{rd} edition), Cold Spring Harbor Laboratory, Cold Spring Harbor Laboratory Press, New York, both of which are incorporated herein by reference in their entirety. Also see, Kunkel, 1985, Proc. Natl. Acad. Sci. 82:488 (describing site directed mutagenesis) and Roberts et al., 1987, Nature 328:731-734 or Wells, J. A., et al. (1985) Gene 34:315 (describing cassette mutagenesis).

Typically, nucleic acids encoding the desired polypeptides are used in expression vectors. The phrase “expression vector” generally refers to nucleotide sequences that are capable of affecting expression of a gene in hosts compatible with such sequences. These expression vectors typically include at least suitable promoter sequences and optionally, transcription termination signals. Additional factors necessary or helpful in effecting expression may also be used as described herein. DNA encoding the polypeptides of the present invention will typically be incorporated into DNA constructs capable of introduction into and expression in an in vitro cell culture. Specifically, DNA constructs will be suitable for replication in a prokaryotic host, such as bacteria, e.g., E. coli, or may be introduced into a cultured mammalian, plant, insect, yeast, fungi or other eukaryotic cell lines.

DNA constructs prepared for introduction into a particular host will typically include a replication system recognised by the host, the intended DNA segment encoding the desired polypeptide, and transcriptional and translational initiation and termination regulatory sequences operably linked to the polypeptide encoding segment. A DNA segment is “operably linked” when it is placed into a functional relationship with another DNA segment. For example, a promoter or enhancer is operably linked to a coding sequence if it stimulates the transcription of the sequence. DNA for a signal sequence is operably linked to DNA encoding a polypeptide if it is expressed as a preprotein that participates in the secretion of the polypeptide. Generally, DNA sequences that are operably linked are contiguous, and, in the case of a signal sequence, both contiguous and in reading phase. However, enhancers need not be contiguous with the coding sequences whose transcription they control. Linking is accomplished by ligation at convenient restriction sites or at adapters or linkers inserted in lieu thereof.

The selection of an appropriate promoter sequence generally depends upon the host cell selected for the expression of the DNA segment. Examples of suitable promoter sequences include prokaryotic, and eukaryotic promoters well-known in the art. See, e.g., Sambrook and Russell (2001, supra). The transcriptional regulatory sequences will typically include a heterologous enhancer or promoter which is recognised by the host. The selection of an appropriate promoter will depend upon the host, but promoters such as the trp, lac and phage promoters, tRNA promoters and glycolytic enzyme promoters are known and available. See, e.g., Sambrook and Russell (2001, supra).

Conveniently available expression vectors which include the replication system and transcriptional and translational regulatory sequences together with the insertion site for the polypeptide encoding segment may be employed. Examples of workable combinations of cell lines and expression vectors are described in Sambrook and Russell (2001, supra). For example, suitable expression vectors may be expressed in, e.g., insect cells, e.g., Sf9 cells, mammalian cells, e.g., CHO cells and bacterial cells, e.g., E. coli.

It will be understood that the H-RSV F or G protein derived (poly)peptides of the invention may be modified to provide a variety of desired attributes, e.g., improved pharmacological characteristics, while increasing or at least retaining substantially all of the biological activity of the unmodified peptide. For instance, the peptides can be modified by extending, decreasing the amino acid sequence of the peptide. Substitutions with different amino acids or amino acid mimetics can also be made.

The individual residues of the immunogenic H-RSV F or G protein derived (poly)peptides of the invention can be incorporated in the peptide by a peptide bond or peptide bond mimetic. A peptide bond mimetic of the invention includes peptide backbone modifications well known to those skilled in the art. Such modifications include modifications of the amide nitrogen, the a-carbon, amide carbonyl, complete replacement of the amide bond, extensions, deletions or backbone cross-links. See, generally, Spatola, Chemistry and Biochemistry of Amino Acids, Peptides and Proteins, Vol. VII (Weinstein ed., 1983). Several peptide backbone modifications are known, these include, ψ [CH₂S], ψ [CH₂NH], ψ [CSNH₂], ψ [NHCO], ψ [COCH₂ ] and ψ [(E) or (Z) CH═CH]. The nomenclature used above, follows that suggested by Spatola, above. In this context, ψ indicates the absence of an amide bond. The structure that replaces the amide group is specified within the brackets.

Amino acid mimetics may also be incorporated in the peptides. An “amino acid mimetic” as used here is a moiety other than a naturally occurring amino acid that conformationally and functionally serves as a substitute for an amino acid in a peptide of the present invention. Such a moiety serves as a substitute for an amino acid residue if it does not interfere with the ability of the peptide to elicit an immune response against the appropriate H-RSV F or G protein derived epitope. Amino acid mimetics may include non-protein amino acids, such as β-, γ-, δ-amino acids, β-, γ-, δ-imino acids (such as piperidine-4-carboxylic acid) as well as many derivatives of L-α-amino acids. A number of suitable amino acid mimetics are known to the skilled artisan, they include cyclohexylalanine, 3-cyclohexylpropionic acid, L-adamantyl alanine, adamantylacetic acid and the like. Peptide mimetics suitable for peptides of the present invention are discussed by Morgan and Gainor, (1989) Ann. Repts. Med. Chem. 24:243-252.

As noted above, the peptides employed in the subject invention need not be identical, but may be substantially identical, to the corresponding amino acid sequence of the H-RSV F or G protein derived epitope as listed in Table 1. Therefore, the peptides may be subject to various changes, such as insertions, deletions, and substitutions, either conservative or non-conservative, where such changes might provide for certain advantages in their use. The peptides of the invention can be modified in a number of ways so long as they comprise a sequence substantially identical (as defined below) to an amino acid sequence of an H-RSV F or G protein derived epitope as listed in Table 1.

Alignment and comparison of relatively short amino acid sequences (less than about 30 residues) is typically straightforward. Optimal alignment of sequences for aligning a comparison window may be conducted by the local homology algorithm of Smith and Waterman (1981) Adv. Appl. Math. 2:482, by the homology alignment algorithm of Needleman and Wunsch (1970) J. Mol. Biol. 48:443, by the search for similarity method of Pearson and Lipman (1988) Proc. Natl. Acad. Sci. (USA) 85:2444, by computerised implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of sequence similarity over the comparison window) generated by the various methods is selected.

The term “sequence identity” means that two polypeptide sequences are identical (i.e., on an amino acid-by-amino acid basis) over a window of comparison. The term “percentage of sequence identity” is calculated by comparing two optimally aligned sequences over the window of comparison, determining the number of positions at which the identical residues occurs in both sequences to yield the number of matched positions, dividing the number of matched positions by the total number of positions in the window of comparison (i.e., the window size), and multiplying the result by 100 to yield the percentage of sequence identity.

As applied to the peptides of the invention, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine.

Pharmaceutical (Vaccine) Compositions and their Administration

The peptides of the present invention and pharmaceutical compositions thereof are useful for administration to mammals, particularly humans, to treat and/or prevent H-RSV infection as well as LRT and other diseases caused by H-RSV infection. Suitable formulations are found in Remington's Pharmaceutical Sciences, Mack Publishing Company, Philadelphia, Pa., 17th ed. (1985), which is incorporated herein by reference.

The immunogenic peptides of the invention are administered prophylactically or to an individual already suffering from the disease. The compositions are administered to a patient in an amount sufficient to elicit an effective immune response to the MHC molecule from which the peptides are derived. An amount adequate to accomplish this is defined as “therapeutically effective dose” or “immunogenically effective dose.” Amounts effective for this use will depend on, e.g., the peptide composition, the manner of administration, the stage and severity of the disease being treated, the weight and general state of health of the patient, and the judgement of the prescribing physician, but generally range for the initial immunisation (that is for therapeutic or prophylactic administration) from about 0.1 mg to about 1.0 mg per 70 kilogram patient, more commonly from about 0.5 mg to about 0.75 mg per 70 kg of body weight. Boosting dosages are typically from about 0.1 mg to about 0.5 mg of peptide using a boosting regimen over weeks to months depending upon the patient's response and condition. A suitable protocol would include injection at time 0, 2, 6, 10 and 14 weeks, followed by booster injections at 24 and 28 weeks.

The pharmaceutical compositions are intended for parenteral, oral or transdermal administration. Preferably, the pharmaceutical compositions are administered parenterally, e.g., subcutaneously, intradermally, or intramuscularly. Thus, the invention provides compositions for parenteral administration which comprise a solution of the immunogenic peptides dissolved or suspended in an acceptable carrier, preferably an aqueous carrier. A variety of aqueous carriers may be used, e.g., water, buffered water, 0.4% saline, 0.3% glycine, hyaluronic acid and the like. These compositions may be sterilised by conventional, well known sterilisation techniques, or may be sterile filtered. The resulting aqueous solutions may be packaged for use as is, or lyophilised, the lyophilised preparation being combined with a sterile solution prior to administration. The compositions may contain pharmaceutically acceptable auxiliary substances as required to approximate physiological conditions, such as buffering agents, tonicity adjusting agents, wetting agents and the like, for example, sodium acetate, sodium lactate, sodium chloride, potassium chloride, calcium chloride, sorbitan monolaurate, and triethanolamine oleate.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the invention, and more preferably at a concentration of 25%-75%. As noted above, the compositions are intended to induce an immune response to the peptides. Thus, compositions and methods of administration suitable for maximising the immune response are preferred. For instance, peptides may be introduced into a host, including humans, linked to a carrier or as a homopolymer or heteropolymer of active peptide units. Alternatively, the a “cocktail” of peptides can be used. A mixture of more than one peptide has the advantage of increased immunological reaction and, where different peptides are used to make up the polymer, the additional ability to induce antibodies to a number of epitopes. For instance, peptides comprising sequences from hypervariable regions of α and β chains may be used in combination. Useful carriers are well known in the art, and include, e.g., thyroglobulin, albumins such as human serum albumin, tetanus toxoid, polyamino acids such as poly(lysine:glutamic acid), influenza, hepatitis B virus core protein, hepatitis B virus recombinant vaccine and the like.

The compositions preferably also include an adjuvant. A number of adjuvants are well known to one skilled in the art. Suitable adjuvants include incomplete Freund's adjuvant, alum, aluminum phosphate, aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion. The effectiveness of an adjuvant may be determined by measuring the amount of antibodies directed against the immunogenic peptide. A particularly useful adjuvant and immunisation schedule are described in Kwak et al. New Eng. J. Med. 327-1209-1215 (1992), which is incorporated herein by reference. The immunological adjuvant described there comprises 5% (wt/vol) squalene, 2.5% Pluronic L121 polymer and 0.2% polysorbate in phosphate buffered saline.

The concentration of immunogenic peptides of the invention in the pharmaceutical formulations can vary widely, i.e. from less than about 0.1%, usually at or at least about 2% to as much as 20% to 50% or more by weight, and will be selected primarily by fluid volumes, viscosities, etc., in accordance with the particular mode of administration selected.

The peptides of the invention can also be expressed by attenuated viral hosts, such as vaccinia or fowlpox. This approach involves the use of vaccinia virus as a vector to express nucleotide sequences that encode the peptides of the invention. Upon introduction into a host, the recombinant vaccinia virus expresses the immunogenic peptide, and thereby elicits an immune response. Vaccinia vectors and methods useful in immunisation protocols are described in, e.g., U.S. Pat. No. 4,722,848, incorporated herein by reference. Another vector is BCG (Bacille Calmette Guerin). BCG vectors are described in Stover et al. (Nature 351:456-460 (1991)) which is incorporated herein by reference. A wide variety of other vectors useful for therapeutic administration or immunisation of the peptides of the invention, e.g., Salmonella typhi vectors and the like, will be apparent to those skilled in the art from the description herein.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990). Both of these references are incorporated herein by reference in their entirety. E.g. transdermal delivery systems include patches, gels, tapes and creams, and can contain excipients such as solubilisers, permeation enhancers (e.g. fatty acids, fatty acid esters, fatty alcohols and amino acids), hydrophilic polymers (e.g. polycarbophil and polyvinyl pyrillidine and adhesives and tackifiers (e.g. polyisobutylenes, silicone-based adhesives, acrylates and polybutene). Transmucosal delivery systems include patches, tablets, suppositories, pessaries, gels, and creams, and can contain excipients such as solubilisers and enhancers (e.g. propylene glycol, bile salts and amino acids), and other vehicles (e.g. polyethylene glycol, fatty acid esters and derivatives, and hydrophilic polymers such as hydroxypropylmethyl cellulose and hyaluronic acid). Enjectable delivery systems include solutions, suspensions, gels, microspheres and polymeric injectables, and can comprise excipients such as solubility-altering agents (e.g. ethanol, propylene glycol and sucrose) and polymers (e.g. polycaprylactones, and PLGA's). Implantable systems include rods and discs, and can contain excipients such as PLGA and polycapryl lactone. Other delivery systems that can be used for administering the pharmaceutical composition of the invention include intranasal delivery systems such as sprays and powders, sublingual delivery systems and systems for delivery by inhalation. For administration by inhalation, the pharmaceutical compositions of the present invention are conveniently delivered in the form of an aerosol spray presentation from pressurised packs or a nebuliser, with the use of a suitable propellant, e.g., dichlorodifluoromethane, trichlorofluoromethane, dichlorotetrafluoroethane, carbon dioxide or other suitable gas. In the case of a pressurised aerosol the dosage unit may be determined by providing a valve to deliver a metered amount. Capsules and cartridges of, e.g., gelatin for use in an inhaler or insufflator may be formulated containing a powder mix of the peptides of the invention and a suitable powder base such as lactose or starch. The pharmaceutical compositions of the invention may be further formulated for administration by inhalation as e.g. described in U.S. Pat. No. 6,358,530.

In another aspect the invention relates to a method for producing a pharmaceutical composition comprising the (poly)peptides of the invention. The method comprises at least the steps of mixing the (poly)peptides of the invention obtained in the methods described above with a pharmaceutically acceptable carrier and further constituents like adjuvant as described above.

### EXAMPLES

1. Material and Methods

1.1. Peripheral Blood Mononuclear Cells (PBMC's)

Buffycoats were obtained from healthy adult blood donors with informed consent. PBMC's were isolated by density centrifugation using lymphoprep (Nycomed Pharma). PBMC's were either thawed from cryopreserved samples (−135° C.), in RPMI 1640 (Gibco), 10% Dimethyl Sulfoxide, 30% fetal bovine serum (FBS, Gibco), or used fresh.

1.2 Virus and Peptides

Human RSV strain A2 (available under number VR 1302 from the ATCC, Manassas, Va., USA) was propagated in HEp-2 cells and titrated by plaque assay. The virus was routinely used in experiments at a MOI of 1 (MOI=multiplicity of infection). A series of 94 peptides, 18 amino acid residues long was synthesised by standard solid phase Fmoc chemistry. All peptides were analysed by mass spectrometry and gave the anticipated (M+H)⁺ except for peptides 11,12 and 85, that were excluded from the study. Peptides 69 and 70 were made at the end of the series and were numbered 95 and 96 respectively. The peptides synthesised were based on the sequence of the fusion protein F of RSV strain A2. They overlapped by 12 amino acid residues. For the G protein we used a series over overlapping peptides representing amino acid residues 161-199 of the G protein of RSV strain A2. The sequences of these G protein-derived peptides were: DFHFEVFNFVPCSI; FEVFNFVPCSICSNN; FNFVPCSICSNNPTCW; SICSNNPTCWAICKRI; SNNPTCWAICKRIPNKKP; WAICKRIPNKKPGKK; and ICKRIPNKKPGKKTT.

1.3 Elispot Assay

96 well filtration plates (MAIPS45 10, Millipore) were coated overnight with anti-IFN-γ coating antibody (Ab) 1-D1K (100 μl, 15 μg/ml, Mabtech) in 0.1M carbonate-bicarbonate buffer pH 9.6, at 4° C. Before adding the cells the plate were washed thoroughly with PBS, blocked for one hour at 37° C. with RPMI 1640 containing 10% FBS. Cells and either virus or peptide were added to the well in a final volume of 200 μl of RPMI 1640, 10% FBS, penicillin and streptomycin. Cells were incubated at 37° C. overnight in a humidified incubator. Then cells were removed by thoroughly washing in PBS and 100 μl of detecting mAb 7-B6-1-biotin (Mabtech), diluted to 1 μg/ml in PBS-0.5% FBS was added to the wells. After incubating 2 hrs at room temperature and washes in PBS, 100 μl (1/1000 diluted in PBS, 0.5% FBS) ExtraAvidine Alkaline Phosphatase Conjugate (Sigma) was added and incubated 1-2 hours at room temperature. Then the plates were washed in PBS and BCIP/NBT substrate was added 100 μl/well, (BCIP/NBT tablets Sigma, 1 tablet diluted in 10 ml H₂O). Spots were counted by two investigators. Most data are represented as the number of spots per 10⁻⁶ PBMC minus background of unstimulated wells. Only in Table 1 the uncorrected number of spots is shown. In the mAb blocking experiments the antibodies were added to the cells prior to the stimulating peptides. The cells and mAb were preincubated for 30 minutes at 37° C., after which the peptides were added to the cultures. The mAb used are culture supernatants of hybridoma's B8.11.2 producing anti HLA-DR and SPVL3 anti HLA-DQ.

2. Immunodominancy of F Protein Derived Peptides

A set of overlapping peptides 18 amino acid residues long and overlapping by 12 amino acid residues, spanning the entire length of the F protein of RSV strain A2 was synthesised. In a preliminary search for antigenic epitopes pools of 6 consecutive peptides were made. These peptide pools were analysed in an elispot assay for their ability to induce IFN-γ production in PBMC from healthy donors. In Table 1 the data are summarised for a group of donors in which the most frequently occurring MHC class II molecules within the caucasian population are represented. We were able to detect IFN-γ production in response to stimulation with intact RSV at a MOI of 1. The level of the response varied from donor to donor but was generally in the order of 100 to 500 spots per 10⁻⁶ PBMC. There was no correlation between better responses and the time of the year the blood was sampled. Most donors responded to more than one peptide pool. Pool four was not recognised by any donor. The peptides represented in this pool cover a stretch of 46 amino acid residues from 121 to 168, partly overlapping the 27 amino acid residue peptide (110-136) that is removed from the fusion protein after furin cleavage in the Golgi-compartment (Gonzalez-Ryes et al., 2001, Proc. Natl. Acad. Sci. USA 98: 9859-9864).

In a second step the individual peptides from the positive pools that were able to induce IFN-γ production were identified for five individual donors (data not shown). For most pools a clearcut response identified one or two positive peptides. Sometimes two adjacent peptides were found positive that possibly contained an overlapping epitope. The peptide length of 18 amino acid residues is sufficiently long to encompass MHC class II peptides and less suitable for MHC class I interaction. For presentation by MHC class I molecules the 18-mer peptides require cleavage to 9-11 amino acid residues in order to be able to bind into the class I binding groove. Yet in our initial screens we used fairly high concentrations of peptides that had not been purified by HPLC. Therefore, it could be possible that the responses we observed were either CD4 or CD8 T cell responses. To determine the T cell subset—CD4 or CD8—responding to the peptides, we performed elispot assays using PBMC depleted for either CD8 T cells or CD4 T cells. For all the peptides that we have tested with subset depleted PBMC we found that the responses where completely abrogated after CD4 depletion (data not shown). The CD4 involvement in the peptide specific responses was further confirmed by peptide stimulations of PBMC in the presence of HLA DR blocking antibody (Ab) B8.11.2 or HLA DQ blocking Ab SPVL3 (data not shown). For most peptides the number of IFN-γ producing cells dropped to zero when the peptide concentration was titrated in the presence of MHC class II blocking Ab B8.11.2 (data not shown). However, in two donors with HLA type DRB1*1302, DR15, DQ6 and DRB1*1301, DQ6 the response against peptides 50 and 51 is DQ restricted (data not shown). These peptides are also recognised in a DQ restricted fashion by a donor expressing HLA alleles DR1, DQ5 (data not shown). A third donor (HLA DR15,9 DQ3,6) recognised two adjacent peptides (peptide numbers 45,46) also in the context of HLA-DQ (data not shown). Another observation made from these experiments is that there appeared to be large differences in the minimal peptide concentration (40 nM-5 μM necessary for optimal T cell activation for the different individual peptides. Yet the fact that we observed the responses means that during a natural infection the total array of peptides that we have detected in the present series of tests can be functionally presented to the immune system, since we could pick up the response without pre-expansion of the T cells in a direct elispot assay. To determine the MHC restriction element for a final set of peptides recognised by donors carrying HLA-DR7,11,15 alleles, antigen presenting cells (APC) were depleted from high responder heterozygous donors. Elispot assays were performed using these APC-depleted PBMC as responders and fixed PBMC from other homozygous donors as APC. It was found that HLA-DR7 presents peptides 6, 10, 39 and 91 and HLA-DR11 presents peptide 39 to CD4 cells.

From the data obtained in the second step described above, we selected a set of 30 peptides that were recognised by at least one donor. In Table 1 we summarise the T cell responses against this set of F peptides for a series of donors in which the most frequently occurring HLA-DR types within the caucasian population are represented (Knipper et al., 2000, Human Immunol. 61: 605-614). For some peptides the possible P1 anchor residue is marked, based on known antigen binding motifs or computer algorithms (Sturniolo et al., 1999, Nature Biotech. 17: 555-561; Rammensee et al., 1997, Molecular Biology Intelligence Unit, MHC ligands and peptide motifs, Springer Verlag, Heidelberg Germany, Int. ISBN 3-540-63125-9).

3. Immunodominancy of G Protein Derived Peptides

We used the same procedure that was employed for the characterisation of immunodominant peptides within the Fusion protein of RSV to characterise immunodominant peptides derived from the G protein of RSV. Because of the high variability of the G protein in different virus isolates we focussed our analysis on a conserved region (amino acid residues 161-199) within G that possibly plays a role in viral attachment to the cell. Within this region we have characterised two peptides (see table 1) that can be recognised by CD4 T cells present in PBMC samples of healthy blood donors.

TABLE 1

| | | | | | |
|---|---|---|---|---|---|
| | | | | SEQ | |
| | Peptide | RSV | | ID | |
| MHC haplotype | number | protein | Aa position | No. | Amino Acid sequence |

| | | | | | |
|---|---|---|---|---|---|
| HLA-DR1 | 2 | F | 7-24 | 3 | KANAITILTAVTFCFAS |
| | 3 | | 13-30 | 4 | TILTAVTFCFASGQNITE |
| | 40 | | 235-252 | 12 | REFSVNAGVTTPVSTYML |
| | 77 | | 457-474 | 20 | YYVNKQEGKSLYVKGEPI |
| HLA-DQ5 | 50 | | 295-312 | 15 | EVLAYVVQLPLYGVIDTP |
| | 51 | | 301-318 | 16 | VQLPLYGVIDTPCWKLHT |
| HLA-DR4 | 2 | F | 7-24 | 3 | KANAITILTAVTFCFAS |
| (DRB1*0401) | 5 | | 25-42 | 5 | GQNITEEFYQSTCSAVSK |
| | 6 | | 31-48 | 6 | EFYQSTCSAVSKGYLSAl |
| | 14 | | 79-96 | 8 | IKQELDKYKNAVTELQLL |
| | 15 | | 85-102 | 9 | KYKNAVTELQLLMQSTPP |
| | 66 | | 391-408 | 18 | YDCKIMTSKTDVSSSVIT |
| | 72 | | 427-444 | 19 | KNRGIIKTFSNGCDYVSN |
| HLA-DR7 | 5 | F | 25-42 | 5 | GQNITEEFYQSTCSAVSK |
| | 6 | | 31-48 | 6 | EFYQSTCSAVSKGYLSAL |
| | 10 | | 55-72 | 7 | SVITIELSNIKENKCNGT |
| | 15 | | 85-102 | 9 | KYKNAVTELQLLMQSTPP |
| | 30 | | 175-192 | 10 | NKAVVSLSNGVSVLTSKV |
| | 39 | | 229-246 | 11 | RLLEITREFSVNAGVTTP |
| | 95 | | 409-426 | 22 | SLGAIVSCYGKTKCTASN |
| | 91 | | 541-558 | 21 | LIAVGLLLYCKARSTPVT |
| HLA-DR9 | 45 | F | 265-282 | 13 | PITNDQKKLMSNVQIVR |
| | 46 | | 271-288 | 14 | KLMSNNVQIVRQQSYSI |
| HLA-DR11 | 39 | F | 229-246 | 11 | RLLEITREFSVNAGVTTP |
| | 57 | | 337-354 | 17 | TDRGWYCDNAGSVSFFPQ |
| HLA-DR13 | 57 | F | 337-354 | 17 | TDRGWYCDNAGSVSFFPQ |
| (DRB1*1301) | | | | | |
| HLA-DQ6 | 50 | | 295-312 | 15 | EVLAYVVQLPLYGVIDTP |
| | 51 | | 301-318 | 16 | VQLPLYGVIDTPCWKLHT |
| HLA-DR15, 51 | 14 | F | 79-96 | 8 | IKQELDKYKNAVTELQLL |
| | 95 | | 409-426 | 22 | SLGAIVSCYGKTKCTASN |
| HLA-DPw4 | | G | 162-175 | 23 | DFHFEVFNFVPCSI |
| (DPB1*0401) | | | | | |
| and | | | | | |
| (DPB1*0402) | | | | | |
| HLA-DR | | G | 177-194 | 24 | SNNPTCWAICKRIPNKKP |

1. A method for ex vivo diagnosis of an MHC class II haplotype-specific immune response to an H-RSV antigen in a subject, comprising the steps of:
(a) determining the MHC class II haplotype of the subject;
(b) incubating peripheral blood mononuclear cells (PBMC's) from the subject with a peptide comprising any one or more of amino acid sequences designated SEQ ID NO:3-SEQ ID NO:24, wherein the amino acid sequence is known to be presented by the MHC class II molecules of the subject as indicated in Table 1, said incubating being under conditions wherein proliferation of, and/or cytokine production by, PBMC's is induced; and,
(c) assaying the proliferation and/or cytokine production.

2. A method according to claim 1, wherein in step (c) the proliferation of, or cytokine production by, T cells is assayed.

3. A method according to claim 2, wherein the T cell proliferation or cytokine production is assayed without pre-expansion of the T cells.

4. A method according to claim 2, wherein proliferation of, or cytokine production by, CD4⁺ T cells is assayed.

5. A method according to claim 4, wherein the cytokine assayed is IFN-γ.

6. A method according to claim 5, wherein IFN-γ production is measured in an Elispot assay.

7. A method according to claim 1, wherein in step (b) the peptide is incubated at a concentration of a least 5 nM.

8. A method according to claim 1, wherein
(a) the subject is one who:
(i) is or was infected with H-RSV or
(ii) has been vaccinated against H-RSV; and
(b) an immune response to an H-RSV antigen measured.

9. A method according claim 8, wherein the a subject has been vaccinated against H-RSV.

10. A method to evaluate the correlation of protection against H-RSV infection with vaccination in a subject, comprising
(a) measuring a response of proliferation or cytokine production to an H-RSV peptide of the subject's PBMC's according to claim 1,
(b) examining the vaccination status of the subject, and
(c) correlating said response in (a) with said vaccination status in (b).

11. A method for immunizing a subject against an H-RSV antigen in an MHC class II haplotype-specific manner, comprising:
(a) determining the MHC class II haplotype of the subject; and,
(b) administering to the subject an immunogenic pharmaceutical composition comprising a peptide comprising any one or more of amino acid sequences designated SEQ ID NO:3-SEQ ID NO:24, wherein the amino acid sequence is known to be presented by the MHC class II molecules of the subject as indicated in Table 1,
thereby immunizing the subject in said MHC class II haplotype-specific manner.

12. A method according to claim 11, wherein the composition is:
(a) formulated for parenteral administration and administered parenterally, or
(b) formulated for transdermal administration and administered transdermally.

13. A method for preventing or treating H-RSV infection in a subject, comprising, before or during said infection, immunizing the subject in accordance with claim 11, thereby preventing or treating said infection.

14. The method according to claim 13 wherein the immunogenic composition is formulated for parenteral or transdermal administration.

15. A method according to claim 3, wherein proliferation of, or cytokine production by, CD4⁺ T cells is assayed.

16. A method according to claim 15, wherein the cytokine assayed is IFN-γ.

17. A method according to claim 3, wherein
(a) the subject is one who:
(i) is or was infected with H-RSV or
(ii) has been vaccinated against H-RSV, and
(b) an immune response to an H-RSV antigen is measured.

18. A method according to claim 17, wherein subject has been vaccinated against H-RSV.

19. A method according to claim 5, wherein
(a) the subject is one who:
(i) is or was infected with H-RSV or
(ii) has been vaccinated against H-RSV, and
(b) an immune response to an H-RSV antigen is measured.

20. A method according to claim 19, wherein subject has been vaccinated against H-RSV.
